(19) United States
(12) Patent Application Publication
(10) Pub. No.: US 2016/0354580 A1
(43) Pub. Date: Dec. 8, 2016

(54) CATHETER DEVICES WITH PLIABLE CATHETER HUBS AND RELATED METHODS

(71) Applicant: B. Braun Melsungen AG, Melsungen (DE)

(72) Inventors: Hui Kuun Teoh, Penang (MY); Mohd Zairizal Bin Zakaria, Penang (MY); Aik Aun Tan, Penang (MY); Chee Mun Phang, Penang (MY); Mohamad Yasin Bin Abdulla, Simpang Ampat (MY)

(21) Appl. No.: 15/170,548

(22) Filed: Jun. 1, 2016

Related U.S. Application Data
(60) Provisional application No. 62/170,032, filed on Jun. 2, 2015.

(51) Int. Cl. A61M 25/00; A61M 5/162; A61M 25/06

(52) U.S. Cl. CPC A61M 25/0097; A61M 25/0606; A61M 5/162; A61M 5/1626; A61M 25/0009

(57) ABSTRACT

A catheter assembly with a catheter, an outer casing positioned over the proximal end and at least part of the hub body of the catheter hub. The outer casing being pliable and has an interior cavity and a septum at a proximal end thereof. A needle hub removably coupled to a proximal end of the catheter hub with the outer casing wedged between the catheter hub and the needle hub. The needle hub has a proximal interior cavity and a shroud with a distal opening. A needle projects distally from the needle hub through the septum of the outer casing and has a needle tip extending out a distal opening of a catheter tube in a ready to use position.

### FIELD OF ART

The present disclosure generally relates to catheter devices and related methods and more particularly to catheter devices having a pliable catheter hub and an optional safety clip with housing and related methods.

### BACKGROUND

After successful catheterization of an intravenous catheter (“IVC”) to a patient, the access site where the catheter tube enters the skin is secured to the patient to prevent the IVC from moving or inadvertently withdrawing from the vessel of the patient. The IVC could stay attached to the patient throughout the hospital stay which could last days. The weight of catheter hub, the weight of the infusion line attached to the catheter hub, and the tightness of the dressing securing the catheter hub to the patient exert a force, while necessary, that can lead to patient discomfort. Furthermore, a catheter hub is typically made of a semi-hard plastic having parting lines and sharp edges which can cause cuts especially for the very thin skin of neonates and geriatrics.

### SUMMARY

The various embodiments of a catheter device with a rubberized catheter hub and an optional safety clip with housing have several features, no single one of which is solely responsible for their desirable attributes. Without limiting the scope of the present embodiments as set forth in the claims that follow, their more prominent features now will be discussed briefly.

Aspects of the present disclosure include a catheter assembly which can comprise a catheter hub having an opening at a proximal end and an interior cavity, a catheter tube extending distally from the catheter hub and communicating with the interior cavity of the catheter hub, an outer casing sleeved over the proximal end of the catheter hub and having an interior cavity and a septum at a proximal end, a needle hub removably coupled to a proximal end of the catheter hub via the outer casing, the needle hub having an interior cavity and a distal end opening, and a needle projecting distally from the needle hub through the septum of the outer casing, and having a needle tip extending out a distal opening of the catheter tube in the ready to use position.

A proximal end of the catheter tube can be attached to a bushing secured in the interior cavity of the catheter hub.

A plurality of detents can extend laterally from a proximal end of the catheter hub and engage with a plurality of slots defined in the outer casing to secure the outer casing to the catheter hub.

The proximal end of the catheter hub can be a female Luer connector with external threads as the plurality of detents.

One or more channels can be formed on the surface of the catheter hub, and one or more ridges can be formed in the interior cavity of the outer casing engaging with the one or more channels of the catheter hub to retain the outer casing to the catheter hub.

The one or more channels can extend circumferentially around the catheter hub.

A flared portion can extend laterally from the catheter hub. The outer casing can extend over the flared portion.

The outer casing can be made of a soft pliable material.

The outer casing can be made of silicone rubber.

The outer casing can form a seal with the proximal end of the catheter hub.

A connector having a male Luer tip can be connected to the proximal end of the catheter hub through the septum of the outer casing.

The septum can have a convex outer surface such that the septum opens outwardly during insertion of the connector.

The outer casing can further comprise a pair of wings extending laterally from an outer surface of the outer casing. The wings can be flexible.

A tab can extend from the outer surface of the outer casing opposite the pair of wings.

The proximal end of the outer casing can be compressed between the proximal end of the catheter hub and a distal end of the needle hub to removably couple the needle hub to the catheter hub.

A clip housing can be disposed in the interior cavity of the needle hub and have an interior cavity, an engaging end, and a distal opening extending through the engaging end and communicating with the interior cavity of the clip housing. A needle guard can be attached inside the interior cavity of the clip housing. The septum of the outer casing can form a seal with the engaging end of the clip housing in a ready to use position, the needle guard can be configured to cover the needle tip in a secured position.

The needle can project distally from the interior cavity of the needle hub through the needle guard and the clip housing, and have a change in profile proximal of the needle tip. The change in profile can abut against a proximal wall of the needle guard to cover the needle tip in the secured position.

The septum of the outer casing can have one or more slits defined therein to form a plurality of flaps, and the engaging end of the clip housing can engage with the one or more slits in the ready to use position. The plurality of flaps can form a seal around the engaging end in the ready to use position.

The clip housing can abut against a shoulder of the interior cavity of the needle hub in the ready to use position.

The engaging end of the clip housing can comprise a cylindrical base and a tip extending from a distal end of the cylindrical base. The tip can be conical shaped.

The needle guard can be fixed to the clip housing.

A proximal end of the outer casing can abut against a shoulder of the interior cavity of the needle hub in the ready to use position.

Another aspect of the present disclosure includes a method of manufacturing a catheter assembly, which can include extending a catheter tube distally from a catheter hub, sleeving an outer casing on the catheter hub, and extending a needle having a needle tip distally through a septum of the outer casing, and the catheter tube in a ready position, wherein the outer casing is flexible.

The method can further comprise extending the needle through a needle guard and a clip housing prior to extending through the septum of the outer casing.

The method can further comprise abutting a change in profile proximal of the needle tip against a proximal wall of the needle guard in a secured position.

The method can further comprise engaging a plurality of slots defined in the outer casing with a plurality of detents extending laterally from a proximal end of the catheter hub to secure the outer casing to the catheter hub.

The proximal end of the catheter hub can be a female Luer connector with external threads as the plurality of detents.

The method can further comprise engaging one or more channels formed on the surface of the catheter hub with one or more ridges formed in the interior cavity of the outer casing to retain the outer casing to the catheter hub.

The one or more channels can extend circumferentially around the catheter hub.

A flared portion can extend laterally from the catheter hub, and the outer casing can extend over the flared portion.

The outer casing can be made of a soft pliable material, such as silicone rubber.

The outer casing forms a seal with the proximal end of the catheter hub.

The method can further comprise connecting a connector having a male Luer tip to the proximal end of the catheter hub through the septum of the outer casing.

The septum can have a convex outer surface opening outwardly during insertion of the connector.

A pair of wings can extend laterally from an outer surface of the outer casing. The wings can be flexible.

A tab can extend from the outer surface of the outer casing opposite the pair of wings.

The method can further comprise removably coupling a distal end of the needle hub to the proximal end of the catheter hub, and compressing the proximal end of the outer casing between the catheter hub and needle hub.

The method can further comprise providing a clip housing and a needle guard attached to the interior cavity of the clip housing.

The clip housing can have an interior cavity, an engaging end, and a distal opening extending through the engaging end and the interior cavity of the clip housing.

The needle guard can be attached inside the interior cavity of the clip housing.

The septum of the outer casing can form a seal with the engaging end of the clip housing in a ready to use position.

The needle guard can be configured to cover the needle tip in a secured position.

The method can further comprise projecting the needle distally from the interior cavity of the needle hub through the needle guard and the clip housing.

The method can further comprise abutting a change in profile proximal of the needle tip against a proximal wall of the needle guard to cover the needle tip in the secured position.

The septum of the outer casing can have one or more slits defined therein to form a plurality of flaps. The engaging end of the clip housing can engage with the one or more slits in the ready to use position. The plurality of flaps can form a seal around the engaging end in the ready to use position.

The method can further comprise abutting the clip housing against a shoulder of the interior cavity of the needle hub in the ready to use position.

The engaging end of the clip housing can comprise a cylindrical base and a tip extending from a distal end of the cylindrical base.

The tip can be conical shaped.

The needle guard can be fixed to the clip housing.

### DESCRIPTION OF DRAWINGS

These and other features and advantages of the present devices, systems, and methods will become appreciated as the same becomes better understood with reference to the specification, claims and appended drawings wherein:

FIG. 1 shows an assembled isometric view of one embodiment of a catheter assembly, the catheter assembly including a catheter hub unit and a needle hub unit;

FIG. 2 shows a cross-sectional side view of the catheter assembly of FIG. 1;

FIG. 3 shows an exploded isometric view of the catheter hub unit of FIG. 1;

FIG. 4A shows a partial cutaway isometric view of an embodiment of an outer casing of the catheter hub unit of FIG. 1;

FIG. 4B shows a partial cutaway isometric view of another embodiment of an outer casing of the catheter hub unit of FIG. 1;

FIGS. 5A-5D show various states of assembling or disassembling of a needle guard and housing onto a needle hub unit; and

FIGS. 6A and 6B shows different states of the catheter assembly when withdrawing the needle hub unit away from the catheter hub unit.

### DETAILED DESCRIPTION

The detailed description set forth below in connection with the appended drawings is intended as a description of the presently preferred embodiments of catheter assemblies for use in various applications provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

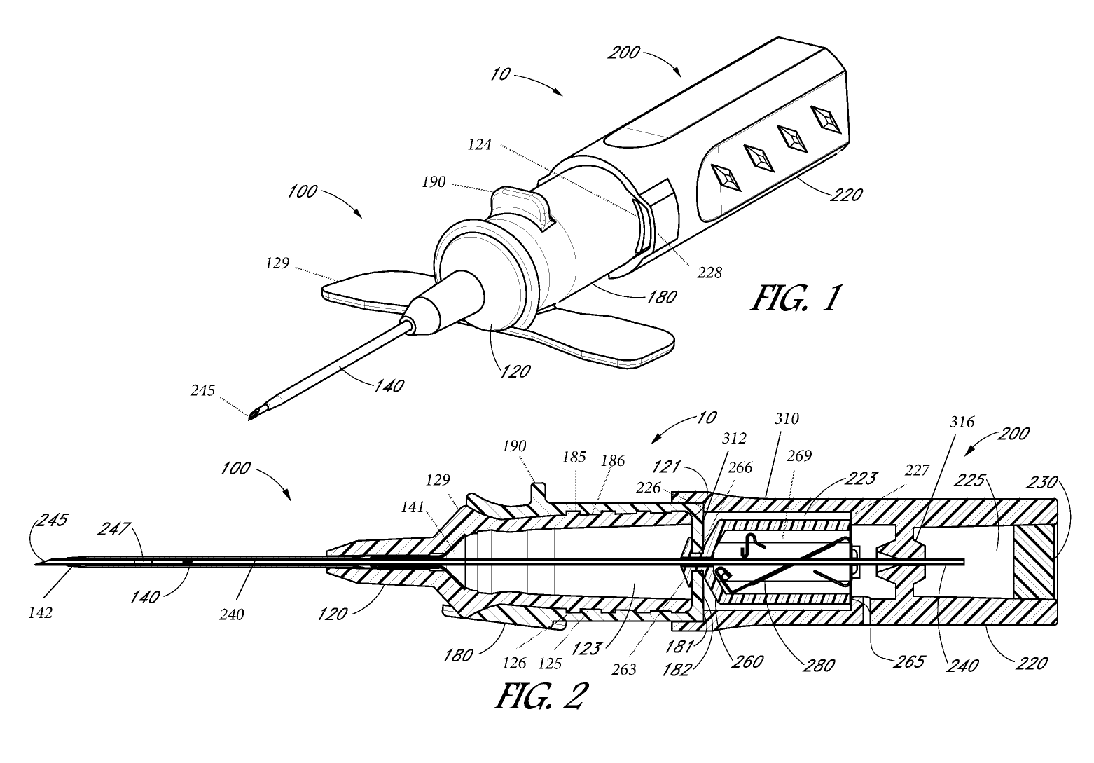

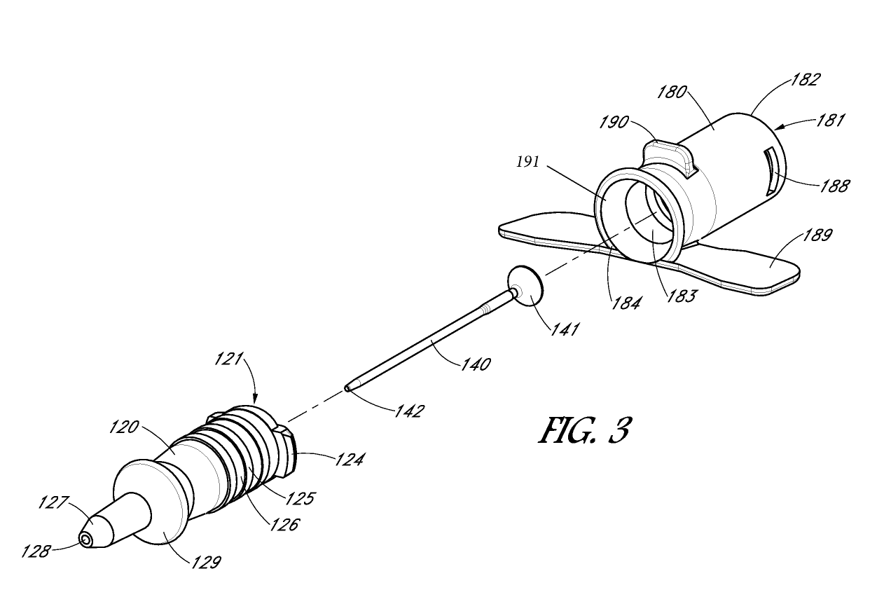

With reference now to Figures (“FIGS.”) 1 and 2, an embodiment of a catheter assembly or catheter device 10 is shown comprising a catheter hub unit 100 and a needle hub unit 200 detachably assembled to a proximal end 121 of the catheter hub unit 100 in a ready to use position. FIG. 3 shows the catheter hub unit 100 of FIGS. 1 and 2 in an exploded view. The catheter hub unit 100 comprises a catheter hub 120 having an interior cavity 123, a catheter tube 140 extending distally from a distal end 127 of the catheter hub 120, and a sleeve or outer casing 180 positioned over, such as encasing or surrounding, the catheter hub 120 from a proximal end 121 of the catheter hub 120. Conventionally speaking, the distal end 127 of the catheter hub 120 is generally toward the patient and the proximal end 121 is generally away from the distal end, typically closer to the practitioner. The needle hub unit 200 comprises a needle hub 220 having a needle 240 extending distally from the needle hub 220 and, in the ready to use position, projects through the catheter hub 120 and the catheter tube 140 with the needle tip extending out a distal end of the catheter tube 140. The needle hub 220 is mechanically coupled to the catheter hub 120, such as by friction, detent, telescoping, some interference fit, or combinations thereof. The needle hub unit 200 can further comprise a clip housing 260 coupled to the outer casing 180 and a needle guard 280 located partially or entirely in the clip housing 260. As further discussed below, the clip housing 260 can mechanically couple to the outer casing 180 and be positioned inside a shroud section of the needle hub 220 or positioned inside the catheter hub 120.

A change in profile 247, such as a bump, a crimp, a bulge, enlarged diameter, or sleeve positioned over the needle shaft, can be incorporated adjacent or near the needle tip 245, such as proximally of the needle tip, and configured to engage the needle guard 280 during withdrawal of the needle 240 to activate the needle guard to secure the needle tip 245 in a protective or secured position. A vent 230 can be provided at the proximal end of the needle hub to allow gas to escape after successful venipuncture so that blood can flow from the patient through the needle 240. For example, the blood can flow towards a proximal interior cavity 225 of the needle hub 220 towards the vent 230, although it should be noted that blood does not need to flow to the proximal interior cavity 225 of the needle hub 220. The vent 230 can be part of a vent plug or blood stopper attached at the proximal end of the needle hub 220.

A distal end of the catheter tube 140 can be inserted into the patient during venipuncture and a proximal end of the catheter tube 140 can be attached to the catheter body 121 by a bushing 141 attached to the distal end 127 inside the interior cavity 123 of the catheter hub 120 to secure the catheter tube 140 to the catheter hub 120. The bushing 141 can also form a seal with the catheter hub 120 to prevent fluid from leaking out the distal end 127 of the catheter hub 120 between the catheter tube and the interior surface. A distal portion 142 of the catheter tube 140 tapers inwardly or has a smaller diameter than the needle 240 to form a seal around the opening of the catheter tube 140 with the shaft of the needle 240 to facilitate entry through the skin when the needle tip 245 accesses the vasculature of the patient. After verification of proper needle 240 and catheter tube 140 placement, retraction of the needle tip 245 in a proximal direction will allow fluid or blood to flow into the annular space between the needle 240 and the interior of the catheter tube 140, known as secondary blood flashback. After the needle hub 220 is removed from the proximal end 121 of the catheter hub 120, an intravenous tubing can be connected to the proximal end 121 of the catheter hub 220 to facilitate delivery of fluids into or removal of fluids from the patient. For example, when the catheter tube 140 is inserted into the patient, fluid or solution, such as saline solution and/or medication, can flow out the fluid source and into the proximal end 121 of the catheter hub 120 and out the catheter tube 140 without leakage.

Exterior threads or a plurality of detents 124 can extend radially or laterally from the exterior surface of the catheter hub 120, such as from the proximal end 121 of the catheter hub 120. The detents 124 of the catheter hub can engage with slots 188 formed at the proximal end 181 of the outer casing 180 to prevent the outer casing 180 from rotating relative to the catheter hub 120 and/or sliding axially from the catheter hub 120, thereby securing the outer casing 180 to the catheter hub 120. The slots 188 of the outer casing 180 can be defined in the outer casing 180 to fit over the detents 124 of the catheter hub 120. The slots 188 can be sized to stretch over the detents 124 or equal to or greater than the size of the detents 124. The proximal end 121 of the catheter hub 120 can be a female Luer with the external threads serving as the detents 124. The female Luer connector of the open proximal end of the catheter hub 120 can be configured to matingly receive a male Luer connector, such as an IV line, a Luer access connector, a syringe tip, a vent plug, other known connectors, or future-developed IV devices. Each of these components can be sized and configured in conformity with at least some of the International Standards Organization (ISO) standards for female and male Luer connections under current or future standards. For discussion purposes, any one of these components or the class of these components may be referred to as a male medical implement.

The outer casing 180 may be made of a soft pliable material capable of stretching, flexing, expanding, or combinations thereof over the catheter hub 120. In one example, the outer casing 180 is made of a silicone rubber or a thermoplastic elastomer. The hardness can be selected to have a soft texture or feel, such as a low durometer, so that when mounted over the catheter hub 120 and then placed against the patient, the contact does not cause or induce scratching or the sensation of biting into the patient generating or causing discomfort. The outer casing 180 has an open distal end 191 that leads to an interior cavity 183. A septum 182 having one or more slits is provided at or near the proximal end 181 of the outer casing 180. The proximal end 181 of the outer casing 180 forms a seal with the proximal end 121 of the catheter hub 120 to prevent fluid from leaking out between the proximal end of the interior cavity 123 of the catheter hub 120 and the outer casing 180. When intended, fluid can also be selectively permitted to flow through the septum 182 at the proximal end. Once the outer casing 180 is installed over the catheter hub 120 and the catheter hub 120 is used in service, the outside of the catheter hub 120 has a relatively softer and pliable feel than traditional molded plastic with parting lines and sharp edges.

One or more ridges 125, channels 126, or both may be incorporated in an outer surface of the catheter hub 120 to matingly engage with a corresponding one or more channels 185 and/or ridges 186 in the interior of the outer casing 180 to prevent the outer casing 180 from shifting axially with respect to the catheter hub 120 and increasing the surface area and thus friction between the catheter hub 120 and the outer casing 180 to prevent rotation or any shifting. Alternatively or in addition thereto, the ridges 125 and/or channels 126 of the catheter hub 120 can be positioned distal of the detents 124, can be equally spaced from each other, and/or can extend circumferentially around the catheter hub 120. Alternatively or in addition thereto, bumps or dimples may be incorporated on the exterior surface of the catheter hub for mating contact with bumps or dimples on the interior of the outer casing 180.

The spacing between adjacent ridges 125 and the width of the ridges 125 and channels 126 of the catheter hub 120 can be any size, length, and extend in any direction such as longitudinally, at an angle, or any regular or irregular shape. In one example, the channels 185 and ridges 186 in the interior of the outer casing 180 are approximately the same size and in matching position with the ridges 125, channels 126, bumps, or dimples, such as forming corresponding mating surfaces, of the catheter hub 120 to firmly secure the outer casing 180 to the catheter hub 120. The engagement between the various channels 126, 185 and corresponding ridges 186, 125 can also prevent contaminants from entering the interior cavity 123 of the catheter hub 120. The size of the interior cavity 181 of the outer casing 180 can be less than or equal to a size of the outer surface of the catheter hub 120 so that the outer casing 180 stretches slightly to fit over the catheter hub 120 and provide a snug fit and form a liquid tight seal.

The catheter hub 120 may also have a flared portion 129, such as an enlarged or oversized portion compared to other nominal cross-sectional area region, extending between the distal end 127 and the proximal end 121 of the catheter hub 120. The flared portion 129 can provide a larger area for a user to grip the catheter hub 120 or for leverage when using the catheter assembly. The outer casing 180 has a wall surface that defines an elongated hollow body so that when coupled to the catheter hub 120 it surrounds the catheter hub 120 from the proximal end 121 of the catheter hub 120 to a point at or just proximal of the flared portion 129. In some instances, the distal end of the outer casing 180 can extend distal of the flared portion 129.

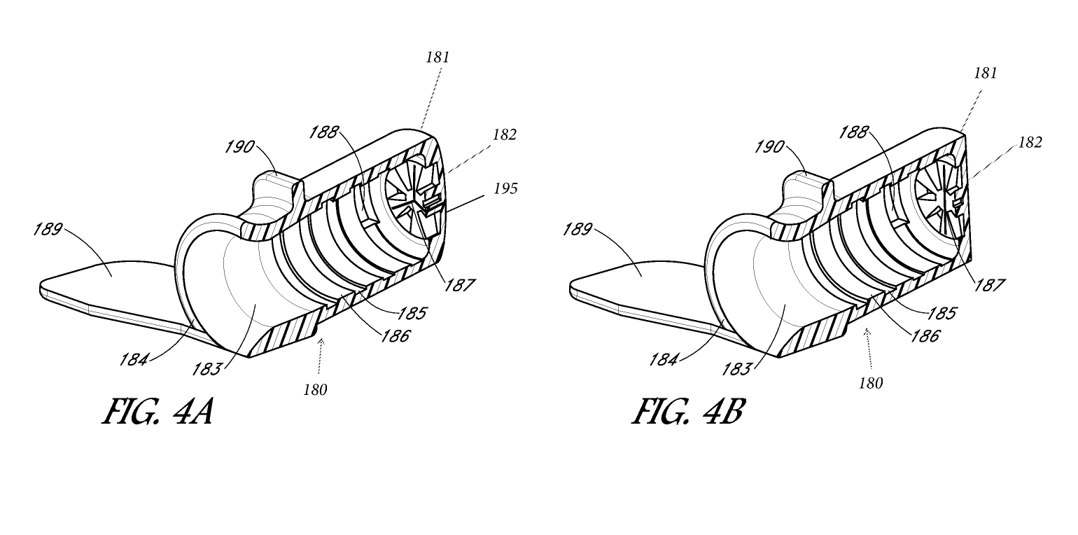

With reference now to FIGS. 4A and 4B in addition to FIGS. 1-3, different embodiments of an outer casing 180 are shown. As shown, the outer casing 180 has an interior cavity 183 and a valve or septum 182 located at a proximal end 181 of the outer casing 180 communicating with the interior cavity 183. The septum 182 of the outer casing 180 can have one or more slits 187 or openings extending through the septum 182, thereby forming two or more flaps. The engaging end 265 of the clip housing 260 can project through the one or more slits 187 of the septum 182 in the ready to use position to engage the septum in the ready to use position. The septum 182 can have cutouts or hollowed out portions 189 defined on an inside surface of the septum 182 between the one or more slits 187. Thus, the one or more slits 187 extend through the thickest portion of the septum 182 separated from an adjacent slip by a hollowed out portion 187, and each flap has a thicker outer perimeter than at a center of the flap. The hollowed out portions 189 between the slits 187 enable the flaps of the hollow inner septum 182 formed by the slits 187 to deflect easily. The slits 187 can form a seal around the engaging end 265 of the clip housing 260 in the ready to use position. If the clip housing 260 is not present, the slits 187 form a seal around the shaft of the needle 240 in the ready to use position. The slits 187 form another seal to prevent fluid such as blood from flowing proximally out of the outer casing 180, after the engaging end 265 of the clip housing 260 or the needle 240 is removed from the septum 182.

The septum 182 is configured to accommodate a male Luer when the same is inserted into the open proximal end of the catheter hub 120 to be in fluid communication therewith. In the illustrated embodiment of FIG. 4A, the septum 182 has a convex outer contour 195 so that during male Luer insertion, the flaps of the septum 182 are forced to open outwardly away from the interior cavity 183. The surface features and materials retained from the cutouts or hollowed out portions 189 on the interior of the flaps create an outward tension so that when the flaps are pushed by a male Luer tip from the proximally facing surface, the flaps deflect outwardly in the proximal direction. In another embodiment shown in FIG. 4B, an exterior surface of the septum 182 is opposite the inside surface of the septum 182 and has a flat surface with one or more slits 187. In either embodiment, the septum 182 is configured to receive a male Luer tip or male medical implement to allow fluid or solution to pass distally into the interior cavity 123 of the catheter hub 120 and out the catheter tube 140 to the patient. For example, when a male medical implement, such as a syringe tip, is inserted through the septum 182, the male medical implement breaks the seal of the septum 182 by opening the one or more slits 187 to allow at least a tip of the male medical implement to pass through into the interior cavity 123 of the catheter hub 120 to open fluid communication therewith.

In another example, the septum 182 is not attached to the proximal end 181 of the outer casing 180 but is located inside the interior cavity 123 of the catheter hub 120 separated from the outer casing 180 and distal from the proximal end 181 of the outer casing 180. In this example, the outer casing 180 would have a proximal opening at the proximal end 181 and the proximal opening allows access to the inlet of the catheter hub, which has a female Luer. The clip housing 260 can then extend partially or entirely into the interior cavity 123 of the catheter hub 120 to engage the septum 182, such as engaging with projections inside the interior cavity 123 of the catheter hub 120. Alternatively, the clip housing 260 can be omitted with the needle guard 280 removably engaged in the interior cavity 123 of the catheter hub 120. Exemplary catheters with a valve being located inside the catheter hub with a needle guard and a valve opener are disclosed in U.S. Pat. No. 8,540,728, the contents of which are expressly incorporated herein by reference.

A pair of wings 189 may extend laterally from an outer surface of the outer casing 180 to provide added surface for stabilization. For example, following catheterization, adhesive may be applied over the wings 189 and the catheter hub to secure and stabilize the catheter hub 120 to the patient. Prior to catheterization, the wings 189 can be used to grip the catheter assembly 10 by bending or flexing the wings 189 towards the catheter hub 120 to provide a grasping surface and for aligning the needle 240 to the puncture site when inserting the needle 240 and catheter tube 140 into the patient. Thus, configuring the wings 189 to be flexible, such as by incorporating notches for folding, by adjusting the thickness and/or the hardness of the material, the wings have functional use before and after venipuncture.

The pair of wings 189 can be placed on top of the skin of the patient adjacent the point of entry of the catheter tube 140 into the patient. The wings 189 may be sufficiently pliable to reduce patient discomfort after successful venipuncture, but not so flexible as to allow significant movement of the catheter tube 140. The wings 189 can extend symmetrically from the outer surface of the outer casing 180. In the illustrated embodiment, the wings 189 extend nearly tangentially from the outer surface of the outer casing 180 adjacent the distal opening 184, thereby forming a flat continuous surface underneath the wings 189. The wings 189 can be secured to the patient by an adhesive, such as on the underside of the wings 189, over the top of the wings 189, or using medical grade dressing to secure the wings 189 and thus the catheter hub 120 to the patient.

The outer casing 180 may include a grip 190 extending radially, laterally, or at an angle from an outer surface of the outer casing 180 to provide a leverage for gripping the catheter assembly 10 during and after venipuncture. The grip 190 can also provide a handle or grip during assembly when stretching the outer casing 180 onto the catheter hub 120.

Because the proximal end 121 of the catheter hub 120 is covered by the outer casing 180, the outer casing 180, such as the convex outer surface 195 and the perimeter of the septum 182, is sandwiched between the catheter hub 120 and the needle hub 220 in the ready to use position. The needle hub 220 can be removably coupled to the catheter hub 120 in the ready to use position by placing the proximal end 181 of the outer casing 280, such as inserting the proximal end of the outer casing 280, into open distal end 191 of the shroud 310 and abutting the outer proximal perimeter 312 of the outer casing against a first shoulder 226 in the shroud 310 of the needle hub 220. Thus, the first shoulder 226 in the distal interior cavity 223 of the shroud 310 of the needle hub 220 can provide a stop for the proximal end 181 of the outer casing 280 to limit the amount of insertion of the proximal end into the distal open distal end 191 of the shroud of the needle hub 220. As shown, the proximal end 121 of the catheter hub 120 is pressed against a distal end 222 of the needle hub 220 with the proximal end 181 of the outer casing 180 compressed between the catheter hub 120 and the needle hub 220, thereby forming a seal between the catheter hub 120 with the needle hub 220. The amount of force required to disengage the coupling between the catheter hub 120 and the needle hub 220 may depend on the frictional force between the needle hub 220 and the outer casing 180, which can depend on the friction coefficient between the outer casing 180 and the needle hub 220 and the magnitude of the compression of the outer casing 180 between the catheter hub 120 and the needle hub 220. Additionally, bumps or other surface features may be added onto the outer casing 180 and or the interior surface of the shroud 310 as interference to increase the separation force. Therefore, the choice of material and compression of the outer casing 180 can determine the pulling force required to overcome the engagement to decouple the needle hub 220 from the catheter hub 120. The needle hub 220 can also form external pockets 228 which engage with the plurality of detents 124 of the catheter hub 120 to secure the needle hub 200 to the catheter hub 120. For example, when assembling the needle hub 220 to the catheter hub 120, the external pockets 228 are slid over the detents 124. This engagement can also prevent rotation of the needle hub 220 with respect to the catheter hub 120.

The needle hub 220 can have a distal interior cavity 223 at the shroud 310 and a proximal interior cavity 225 separated by a wall 316, which includes a needle well for attaching the needle to the needle hub 220. The proximal interior cavity 225 forms a primary flashback chamber and collects fluids, such as blood, passing through the needle 240 and into the chamber. The clip housing 260 can be disposed partially or entirely in the distal interior cavity 223 of the shroud 310 of the needle hub 220. The clip housing 260 can fit loosely within the shroud 310 or can touch the interior cavity 224 of the shroud 310 with the engagement end 265 of the clip housing 260 secured to the septum 182 of the outer casing 180. The spring clip or needle guard 280 can be located inside an interior cavity 269 of the clip housing 260. The needle 240 can extend distally from the distal interior cavity 223 of the needle hub 220, through the needle guard 280, the clip housing 260, the outer casing 180, the catheter hub 120, and out through a distal opening of the catheter tube 140 in the ready to use position with the needle tip 245 of the needle 240 extending distally of the distal opening of the catheter tube 140.

The clip housing 260 is configured to support the needle guard 280, such as to hold or accommodate the needle guard in the ready to use position. The clip housing 260 can provide a means for retaining the needle guard 280 to the catheter hub 120 in the ready to use position and during retraction of the needle 240 from the catheter tube 140 and the catheter hub 120 following successful venipuncture. As previously described, the means for retaining can comprise an engaging end 265 extending distally of the clip housing 260 and projecting through the one or more slits of the septum 182 to engage the septum.

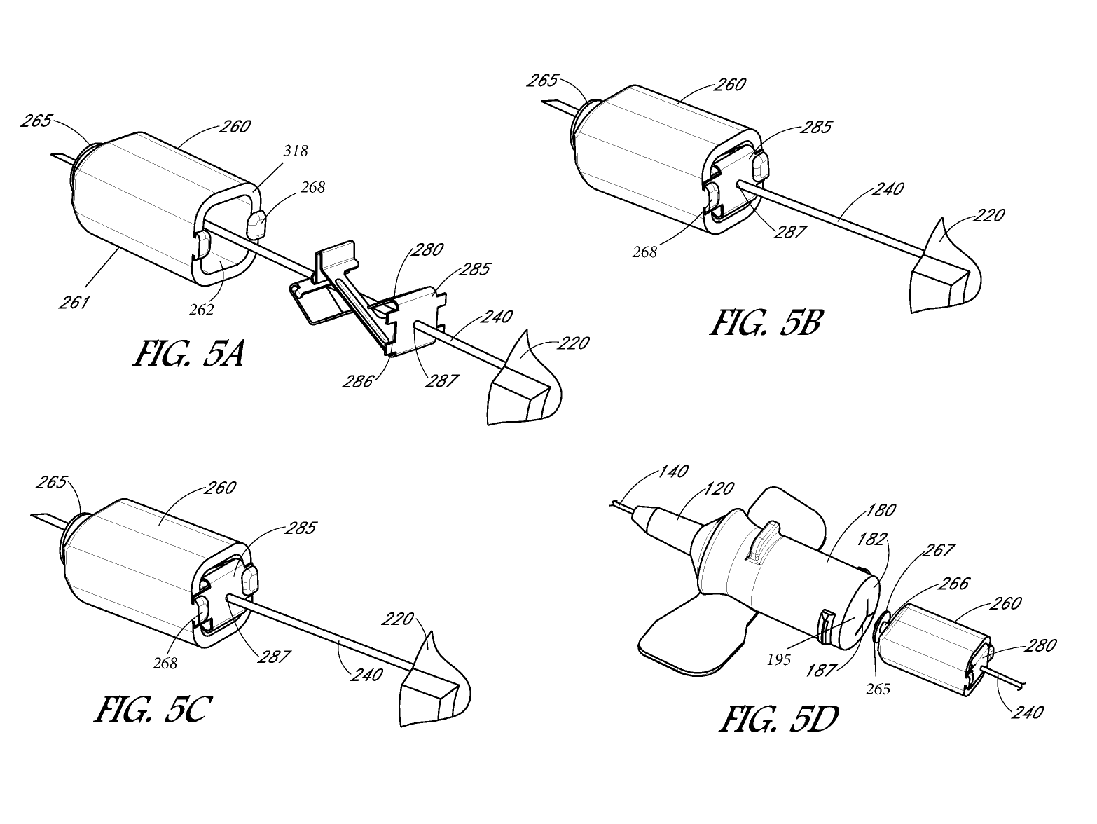

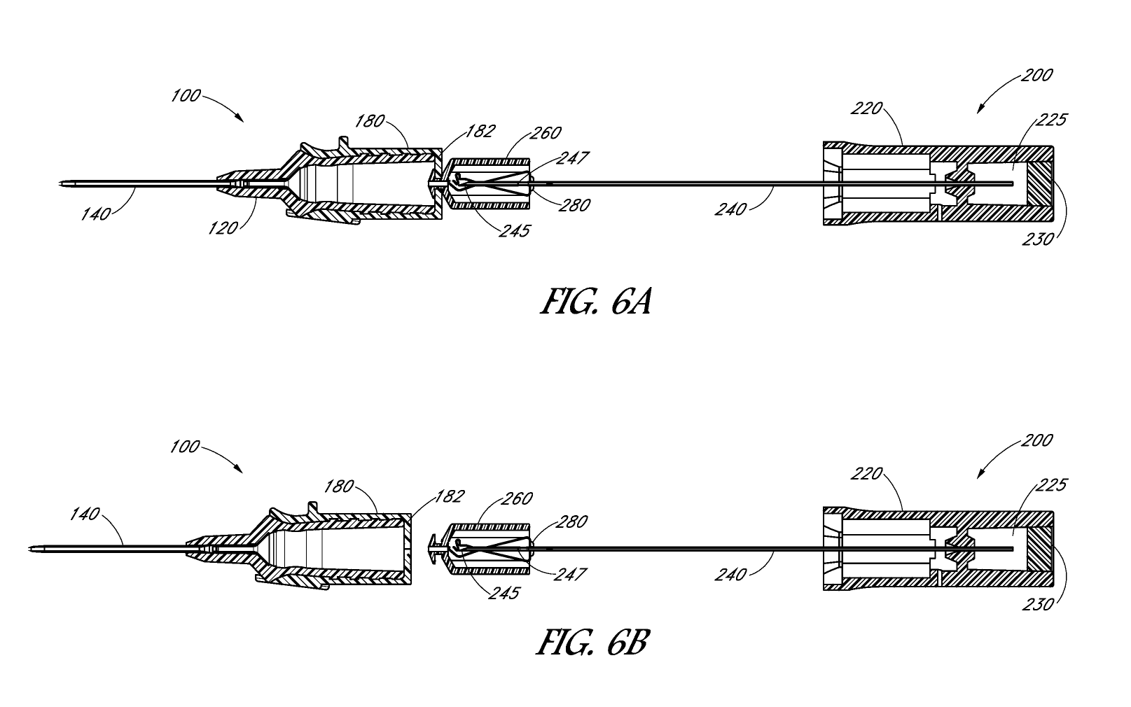

With reference to FIGS. 5A-5D, the clip housing 260 has a wall surface defining a housing body 261, a proximal opening 262, an interior cavity 269, an engaging end 265 extending distally from a distal end of the housing body 261, and a distal opening 263 through the engaging end 265 for accommodating the needle. The distal opening 262 is in communication with the proximal opening 262 through the interior cavity 269. One or more mounting bosses 268 extend proximally from the proximal end perimeter of the housing body 261. A proximal end of the needle guard 280 is configured to engage the housing body 261, such as the one or more bosses 268, to fix the needle guard relative to the clip housing along an axial direction.

Once mounted inside the distal cavity 223 of the shroud 310, the clip housing 260 can abut against a second shoulder 227, proximal of the first shoulder 226. The distance between the first shoulder 226 and the second shoulder 227 should be greater than or equal to the proximal end 318 of the housing body 261 and the interface between the end engaging end 265 and the proximal end 181 of the outer casing 180. That is, the clip housing 260 should be able to fit inside the distal interior cavity 223 of the shroud 310 without exerting any or without exerting a noticeable axial force against the septum of the outer casing 180 to ensure that the septum 182 is not deflected by the axial force. This also ensures a seal with the engaging end 265 of the clip housing 260.

In an example, the engaging end 265 can comprise a conical shaped tip 267 extending from the distal end of the clip housing 260, a generally planar or flat proximal surface, and a cylindrical base 266. The engagement end 265 can resemble a mushroom head having a lumen extending therethrough and the cylindrical base 266 resembles a stem extending from the mushroom head. In one example, the engaging end 265 is fully engaged with the septum 182 of the outer casing 180 without deflecting the septum 182 and abutting against the second shoulder 227 of the needle hub 220. The distal opening 263 of the clip housing 260 should be sized to be slightly larger than the size of the change in profile 247 on the needle to ensure that the needle tip 245 passes through the distal opening 263 to be captured by the needle guard 280.

FIGS. 5A-5D may be viewed as illustrating a method of forming a guard and guard housing and the needle hub unit 200. Referring again to FIG. 5A, the needle 240 extends distally from the distal end of the needle hub 220, through the needle guard 280 and the clip housing 260. The change in profile 247 (shown in FIG. 2), such as a crimp, bulge, sleeve, or enlarged diameter, near the needle tip 245 of the needle 240 engages with a hole 287 of the needle guard 280. The change in profile 247 can be formed after passing through the needle guard 280 or before by placing the proximal end of the needle shaft through the guard from the distal end towards the proximal end of the needle guard.

The needle guard 280 can embody a number of different guard devices or configurations for covering and securing the needle tip 245 from inadvertent needle sticks after withdrawing the needle hub 220 from the catheter tube and the catheter hub following successful venipuncture. Exemplary needle guards 280 useable with the present clip housing 260 can be found in U.S. Pat. No. 8,827,965 and as the needle safety element presented in U.S. patent application Ser. No. 13/257,572, published as US 2012/0046620 A1, the contents of which are expressly incorporated herein by reference. The needle guard 280 is attached to the clip housing 260. In one embodiment, the needle guard 280 and the clip housing 260 are integrally formed. In the illustrated embodiment, the clip housing 260 has a housing shape that is polygonal with four sides and four rounded corners. In another embodiment, the clip housing 260 is generally cylindrical but can embody other shapes that can fit inside the distal interior cavity 223 of the shroud 310.

Referring to FIG. 5B, a distal portion of the needle guard 280, which includes two resilient arms and two distal walls at the ends of the two resilient arms, is configured to project into the interior cavity 261 of the clip housing 260, and a proximal wall 285 of the needle guard 280 is attached to the engaging end 265 of the clip housing 260. In the illustrated embodiment, two mounting bosses 268 extend proximally from the clip housing 260 to slidably engage with cutouts 286 at the proximal wall 285 of the needle guard 280, which has a perimeter defining a proximal opening 287 having a smaller opening dimension than the largest dimension of the change in profile on the needle 247 so that the change in profile can engage the perimeter of the proximal wall 285 with the proximal opening 287 to retract the needle guard and the clip housing away from the catheter hub following successful venipuncture. In other examples, the needle guard 280 is joined to the clip housing 260 by other methods, such as by fasteners, tabs fitted into slots, or any conventional securing mechanisms.

Referring to FIG. 5C, the needle guard 280 is shown attached to the clip housing 260. In the illustrated embodiment, the mounting can be made more secured with the bosses 268 by melting them or deforming them, such as by using a heat source to create an interference, to secure the proximal wall 285 of the needle guard 280 to the clip housing 260.

Referring to FIG. 5D, after the needle guard 280 is firmly attached to the clip housing 260 or at least prevented from moving proximally with respect to the clip housing 260, the needle hub unit 200 can be connected to the catheter hub unit 100 by inserting the engaging end 265 if the clip housing into the septum 182 until the flaps of the septum 182 slip between, for example, the conical shaped tip 267 and the distal end of the clip housing 260 and seals around, for example, the cylindrical base 266.

Referring to FIG. 6A, after successful venipuncture, the needle hub 220 can be retracted proximally from the catheter hub 120. The distal walls at the ends of the resilient arms of the needle guard 280 are biased against the needle 240 in the ready to use position. As the needle hub 220 is retracted, the needle 240 is pulled out through the catheter tube 140, into the interior cavity 123 of the catheter hub 120 and continues until the distal walls of the needle guard 280 are no longer biased against the needle 240 and, due to the resilient arms, spring closed to block the needle tip 245. The needle tip 245 enters proximally of the two distal walls on the two resilient arms of the needle guard 280 and the change in profile 247 of the needle 240 abuts against the proximal wall 285 of the needle guard 280. At this point, the needle tip 245 is secured from unintended needle sticks

Referring to FIG. 6B, because the size of the proximal opening 287 on the proximal wall 285 of the needle guard 280 through which the needle 240 passes through is smaller than a largest dimension of the change in profile 247 on the needle 240, when the change in profile 247 abuts against the proximal wall 285 of the needle guard 280, further retraction of the needle hub 220 causes the change in profile 247 to pull on the needle guard 280 and hence the clip housing 260 in the proximal direction away from the septum 182. The septum 182 then seals upon itself to form a seal thereby preventing blood from flowing proximally out through the septum 182. When retracted, the needle tip 245 is secured in both the needle guard 280 and the clip housing 260 and blood droplets, if any, at the needle tip 245 can be captured by the clip housing 260. Thus, the clip housing also serves to at least capture some blood that may drip from the needle tip 245.

Methods of making and of using the catheter assemblies with securing mechanisms and their components as discussed elsewhere herein are within the scope of the present invention.

Although limited embodiments of various catheter assemblies having a pliable outer casing or sleeve have been specifically described and illustrated herein, many modifications and variations will be apparent to those skilled in the art. For example, any over the needle catheter assemblies can benefit by using the securing mechanism disclosed herein to provide quicker attachment to the patient. Furthermore, it is understood and contemplated that features specifically discussed for one catheter assembly with pliable outer casing may be adopted for inclusion with another catheter assembly provided the functions are compatible. Accordingly, it is to be understood that the catheter assemblies and their components constructed according to principles of the disclosed devices, systems, and methods may be embodied other than as specifically described herein. The disclosure is also defined in the following claims.

1. A catheter assembly, comprising:
a catheter hub having a hub body, an opening at a proximal end, and an interior cavity defined by the hub body;
a catheter tube extending distally from the catheter hub and communicating with the interior cavity of the catheter hub;
an outer casing positioned over the proximal end and at least part of the hub body of the catheter hub, said outer casing having an interior cavity and a septum at or near a proximal end;
a needle hub removably coupled at the proximal end of the catheter hub such that part of the outer casing is wedged between the needle hub and the catheter hub, the needle hub having interior proximal cavity and a distal cavity; and
a needle projecting distally from the needle hub through the septum of the outer casing and having a needle tip extending out a distal opening of the catheter tube in a ready to use position.

2. The catheter assembly of claim 1, wherein a plurality of detents extend laterally from the proximal end of the catheter hub engaging with a plurality of slots defined in the outer casing to secure the outer casing to the catheter hub.

3. The catheter assembly of claim 2, wherein the proximal end of the catheter hub is shaped as a female Luer with external threads, the external threads being the plurality of detents.

4. The catheter assembly of claim 3, wherein one or more channels are formed on an exterior surface of the catheter hub, and one or more ridges are formed in the interior cavity of the outer casing engaging with the one or more channels of the catheter hub to retain the outer casing to the catheter hub.

5. The catheter assembly of claim 1, wherein a flared portion extends laterally from the catheter hub, the outer casing extends over the flared portion.

6. The catheter assembly of claim 1, wherein the outer casing is made of a soft pliable material.

7. The catheter assembly of claim 6, wherein the outer casing forms a seal with the proximal end of the catheter hub.

8. The catheter assembly of claim 1, wherein a connector having a male Luer tip is connected to the proximal end of the catheter hub through the septum of the outer casing.

9. The catheter assembly of claim 1, wherein the septum has a plurality of slits defining a plurality of flaps and a convex outer surface.

10. The catheter assembly of claim 9, wherein the convex outer surface creates an outward tension so that when the flaps are pushed by a male Luer tip, the flaps defect in a proximal direction.

11. The catheter assembly of claim 1, wherein the septum is positioned inside the interior cavity of the catheter hub, and the outer casing has a proximal opening at the proximal end.

12. A method of forming a catheter assembly, the method comprising:
extending a catheter tube in a distal direction of a catheter hub having a body;
placing an outer casing over at least part of the body of the catheter hub; and
extending a needle having a needle tip distally through a septum formed with the outer casing and out a distal end of the catheter tube in a ready position; and
wherein the outer casing is flexible.

13. The method of claim 12, further comprising extending the needle through a needle guard.

14. The method of claim 13, further comprising placing the needle guard inside a clip housing, said clip housing located in a distal interior cavity of a shroud.

15. The method of claim 12, further comprising engaging a plurality of slots formed in the outer casing with a plurality of detents extending laterally from a proximal end of the catheter hub to secure the outer casing to the catheter hub.

16. The method of claim 15, wherein the proximal end of the catheter hub is shaped with a female Luer with external threads.

17. The method of claim 12, wherein a flared portion extends laterally from the catheter hub and the outer casing extends over the flared portion.

18. The method of claim 12, wherein the outer casing is made of a soft pliable material.

19. The method of claim 18, wherein the outer casing forms a seal with the proximal end of the catheter hub.

20. The method of claim 12, wherein the septum has a convex outer surface.
